(19) United States
(12) Patent Application Publication
(10) Pub. No.: US 2002/0099442 A1
(43) Pub. Date: Jul. 25, 2002

(54) Body part substitute and method of mounting the same

(76) Inventor: Maria Niino, Tokyo (JP)

Correspondence Address: Felix J. D'Ambrosio, JONES, TULLAR & COOPER, P.C., P.O. Box 2266 Eads Station, Arlington, VA, 22202, US

(21) Appl. No.: 10/035,312

(22) Filed: Jan. 4, 2002

(30) Foreign Application Priority Data
Jan. 19, 2001 (JP) 2001-049426

(51) Int. Cl. A61F 2/52; A61F 2/50; A61F 2/54

(52) U.S. Cl. 623/7; 623/27; 623/57

(57) ABSTRACT

It is sought to provide a body part substitute, which can be held mounted without feel of uncomfortability and be held mounted continuously for long time without possibility of occasional detachment, and a method of mounting the same. A thin film having breathable property, flexibility and extensible-contractible property is attached to the corresponding portion of the body part substitute to be held in close contact with the human body. Alternatively, the thin film is bonded to the human body, and then the corresponding portion of the body part substitute is bonded to the thin film. By preparing the thin film as a transparent film, the skin of the human body can be seen through the thin film, and more natural feel is thus obtainable.

### BACKGROUND OF THE INVENTION

1. Field of the Invention

This invention relates to a body part substitute such as an artificial breast and a method of mounting the same.

2. Description of the Prior Art

When resection of the breast is done in surgical operation, an artificial breast may be mounted on the site. Also, when a finger is accidentally cut off, an artificial finger may be mounted. Further, when any other parts of the human body are lost, then such lost parts may be desired to be made up by suitable artificial substitutes.

Heretofore, body part substitutes such as artificial breasts and artificial fingers were mostly made of viscous silicone or like material, as disclosed in, for instance, Japanese Patent Disclosures Nos. 6-154169, 6-121806, 6-121807, 10-128870 and 2000-175949. As shown in Japanese Patent Disclosure 2000-176002, the producing technique has been advanced such that the body part substitute is almost incapable of discriminating it in flexibility, feel, color and so forth from the actual body part. In fact, it is possible to permit reproduction of real expression of blood vessels and blue veins as seen through the skin. It is also possible to permit reproduction of expression of color changes such as the body part substitute becomes reddish when dipped in hot water.

Such body part substitutes, however, have a problem that they can not be held mounted on the human body for long time.

Heretofore, a body part substitute which is made of viscous silicone such as to resemble the pertinent body art is applied to the human body by utilizing its viscosity. When the body part substitute is applied in this way, however, sweat generated on the skin on the mounted site is not exhaled and produces uncomfortability. Besides, the sweat weakens the adhesion, thus giving rise to the possibility of occasional detachment, that is, it is a problem that the body part substitute can not be held mounted on the human body continuously for long time.

Japanese Patent Disclosure No. 9-276306 discloses an artificial breast, in which a medical adhesive film is applied to part of its body to be held in close contact with the body for alleviating the burden on the skin. Even with this technique, however, it has been impossible to solve the problem that the body part substitute is detached from the human body in a short period of time.

### SUMMARY OF THE INVENTION

The invention was made for solving the above problem, and it has an object of providing an artificial substitute, which can be mounted on the human body without feel of uncomfortability and be continuously held mounted on the human body for long time without possibility of occasional detachment, and also a method of mounting the same.

The body part substitute according to the invention has a structure in which a thin film has breathable property, flexibility and extensible/contractible property and is bonded to the corresponding portion thereof that is to be held in close contact with the skin of the human body.

By preparing the thin film as a transparent film, the skin can be seen through the thin film, and more natural feel is thus obtainable.

In one method of mounting the body part substitute according to the invention, the film having the breathable property, flexibility and extensible-contractible property is bonded to a corresponding portion of the body part substitute that is to be held in close contact with the skin, and then the thin film is bonded to the human body. In an alternative method, the film is first bonded to the human body, and then the corresponding portion of the body part substitute is bonded to the thin film.

In either case, by preparing the thin film as a transparent film the skin of the human body can be seen through the thin film, and more natural feel is obtainable.

### BRIEF DESCRIPTION OF THE DRAWINGS

These and other objects, features and advantages of the invention will become more apparent upon reading of the following detailed specification and drawings, in which:

FIG. 1 is a sectional view showing an embodiment of an artificial breast as a body part substitute according to the invention;

FIG. 2 is a sectional view showing a method of mounting the artificial breast;

FIG. 3 is a sectional view showing a different method of mounting the artificial breast; and

FIG. 4 is a sectional view showing a different embodiment of an artificial breast as a body part substitute according to the invention.

### DETAILED DESCRIPTION OF THE REFERRED EMBODIMENTS

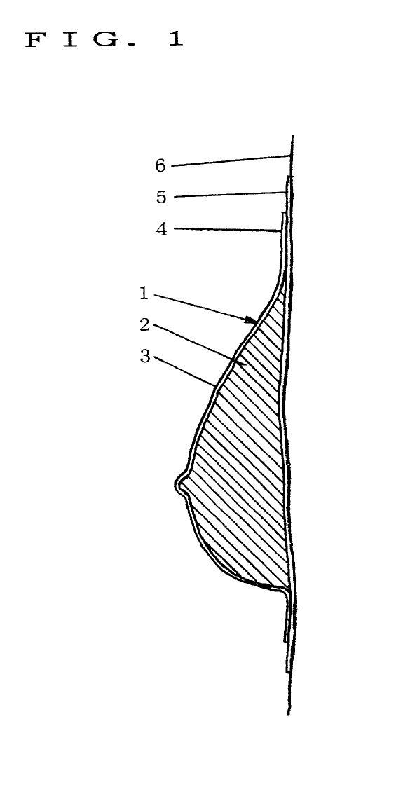

FIG. 1 is a sectional view showing an embodiment of a body part substitute according to the invention applied to an artificial breast 1.

The illustrated artificial breast 1 has a structure, which has a main part 2 of the artificial breast body 1, which is made of a gel-like viscous silicone resin and having a shape of the breast, and a thin silicone film 3 having a thickness of 1 mm or below, which covers the outer surface of the main part 2 and has a marginal portion extending from the edge of the artificial breast main part 2 to form a wind part 4. The silicone film 3, particularly the wing part 4 thereof, is desirably transparent. A thin transparent film 5 having breathable property, flexibility and also extensible/contractible property and also having a thickness of 1 mm or below, is attached on the corresponding portion of the artificial breast 1 in close contact with the skin of the human body. The wing part 4 and the thin transparent film 5 are bonded together with an adhesive, while the main part 2 and the thin transparent film 5 are bonded together with the adhesive property of the material (i.e., adhesive silicone resin) itself of the main part 2.

For the thin transparent sheet 5, such a protective film as Tegaderm Transparent Dressing (trade name by 3M Health Care Co., Ltd.), which is used for observing the state of recovery of such site as a suture after surgical operation or a wound by holding it applied directly on the site is best suited. Such a protective sheet is strongly adhesive enough to hold it applied until healing of the wound and is also transparent, so that it has an effect that the site with the body part substitute applied thereto looks so natural and does not seem that it is applied.

The artificial breast 1 as described above may be attached on the human body in one of two different methods.

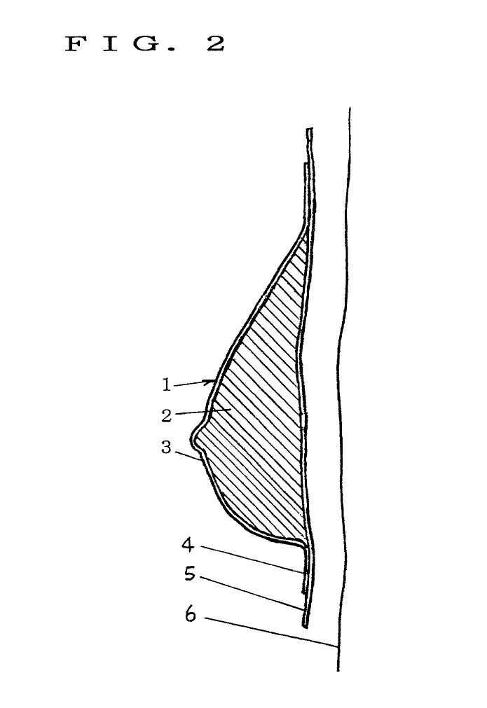

In one such method, as shown in FIG. 2, the thin transparent film 5 is first bonded to the corresponding portion of the artificial breast 1 that is to be held in close contact with the skin. Then the film 5 is applied to the skin 6 of the human body by using such adhesive as acrylic acid adhesive, for instance Darmabond (a trade name).

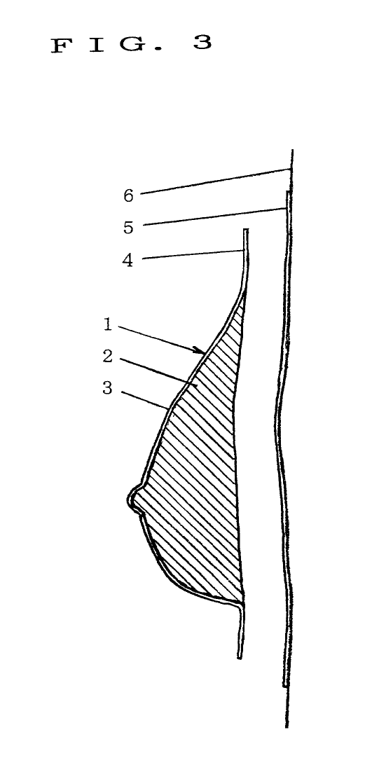

In the other method, as shown in FIG. 3, the thin transparent film 5 is first applied to the skin 6 of the human body by using the adhesive. Then, the wind part 4 of the artificial breast 1 is bonded to the film 5 by using the adhesive.

By attaching the artificial breast 1 on the human body in the above way, sweat generated in the mounted site is exalted through the thin transparent film 5, owing to the breathable property thereof, which allows good oxygen and moisture vapor exchange. Thus, the body part substitute can be mounted without feel of any uncomfortability, and also its mounting produces comfortability. In addition, sweat on the skin surface is quickly dried, it is not possible that the effect of bonding between the thin transparent sheet 5 and the skin is spoiled by sweat. Thus, the artificial breast 1 once attached is not undesirably detached from the human body, and permits sports, bathing and so forth while it is held mounted. Furthermore, the thin transparent film 5 does not stand out, and more natural feel is obtainable.

In the above embodiment, the thin transparent film 5 is described to have a larger size than that of the artificial main part 2 so that the film 5 is extended from the wing part 4 as shown in FIGS. 1 and 2.

However, the thin film 5 may be used to have a smaller size than that of the main part 2 so that the thin film 5 is not extended from the wing part 4 and is thus hidden by the main part 2 when the thin film 5 is attached to the human body. In this connection, the wing part 4 can be deleted totally or at least partially.

Further, it is also possible to use a more smaller sized thin film 5 which is attached only to the central portion of the main part 2 since a good effect of bonding can be maintained between the thin film 5 and the skin 6 of the human body.

While the above embodiment of the body part substitute concerned with the artificial breast 1, it is possible to produce any body part substitute as desired, such as artificial fingers, artificial ears, artificial noses and other losses as well. Any body part substitute, like the artificial breast 1, features the bonding of the thin film 5 having the breathable property, flexibility and extensible/contractible property on its part to be held in close contact with the human body.

It is further possible to produce the body part substitute according to the invention as a plurality of component parts.

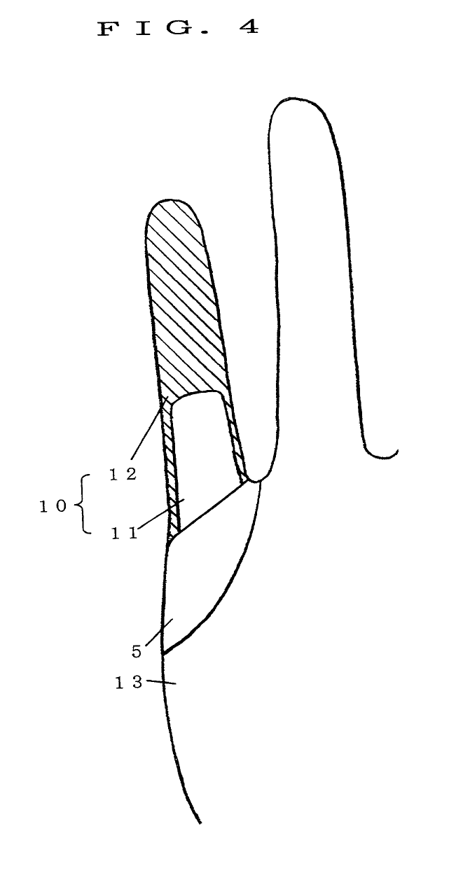

As an example, as shown in FIG. 4, an artificial finger 10 for treating a loss of finger may be produced as the combination of an artificial bone part 11 and an outer part 12.

In this case, the artificial bone part 11 is first firmly secured via the thin transparent film 5 to the human body 13, and then the outer part 12 is mounted on the outside of the bone part 11. Of course, the artificial bone part 11 and the outer part 12 may be more firmly bonded together by interposing the thin transparent film 5 between them.

As has been described in the foregoing, according to the invention since the body part substitute according to the invention is attached on the human body via the thin film having the breathable property, flexibility and extensible/contractible property, sweat can be efficiently exhaled owing to the breathable property of the thin transparent film. Thus, the body part substitute may be mounted without feel of any uncomfortability, and its mounting produces comfortability. In addition, the effect of bonding between the thin transparent film 5 and the skin can be continuously maintained without any possibility of undesired detachment of the body part substitute from the human body.

Furthermore, the thin transparent film does not stand out, and more natural feel is obtainable.

What is claimed is:

1. A body part substitute comprising a thin film having breathable property, flexibility and extensible/contractible property, the thin film being bonded to the corresponding portion of the body part substitute to be held in close contact with the human body.

2. The body part substitute according to claim 1, wherein the thin film is a transparent film.

3. A method of mounting a body part substitute comprising the steps of bonding a thin film having breathable property, flexibility and extensible/contractible property to the corresponding portion of the body part substitute to be held in close contact with the human body, and then bonding the thin film to the human body.

4. The method of mounting the body part substitute according to claim 3, wherein the thin film is a transparent film.

5. A method of mounting a body part substitute comprising the steps of bonding a thin film having breathable property, flexibility and extensible/contractible property to the human body and then bonding the corresponding portion of the body part substitute to the thin film.

6. The method of mounting the body part substitute according to claim 5, wherein the thin film is a transparent film.
